(19) United States
(12) Patent Application Publication
(10) Pub. No.: US 2026/0033758 A1
(43) Pub. Date: Feb. 5, 2026

(54) ELECTRODE ARRANGEMENTS FOR ANALYTE MONITORS

(71) Applicant: Laxmi Therapeutic Devices, Inc., Goleta, CA (US)

(72) Inventors: William Peter Van Antwerp, Santa Clarita, CA (US); Thomas Metzmaker, Goleta, CA (US); Ian Casson, Santa Barbara, CA (US); Timothy J. Uhl, Goleta, CA (US); Landon Christopher Peik, Santa Barbara, CA (US)

(21) Appl. No.: 19/288,661

(22) Filed: Aug. 1, 2025

Related U.S. Application Data
(60) Provisional application No. 63/678,921, filed on Aug. 2, 2024.

(51) Int. Cl. A61B 5/1486; A61B 5/145

(52) U.S. Cl. CPC A61B 5/14865; A61B 5/14532; A61B 2562/06

(57) ABSTRACT

A monitor for determining analyte concentrations in vivo includes a housing configured to adhere to a patient's skin, and a sensor member configured to extend from the housing into the patient's skin. The sensor member includes at least a first working electrode and a separate second working electrode that are independently connected to electronics in the housing via separate electrical connections and that function independently from one another.

### CROSS-REFERENCE TO RELATED APPLICATION(S)

The present application claims priority to and the benefit of both U.S. Provisional Patent Application No. 63/678,921, filed Aug. 2, 2024, the contents of which are hereby incorporated by reference in their entirety.

### BACKGROUND

### Field

The present disclosure relates to medical devices such as analyte sensors, which can be used to measure analytes, such as glucose levels, in the body of a subject. More specifically, the present disclosure is directed to improvements in sensor members associated with such medical devices, as well as methods of use and methods of manufacturing such improved sensor members.

### Description of Related Art

Monitoring different analytes in the human body can be used for various diagnostic reasons. In particular, monitoring glucose levels is important for individuals suffering from type 1 or type 2 diabetes. People with type 1 diabetes are unable to produce insulin or produce very little insulin, while people with type 2 diabetes are resistant to the effects of insulin. Insulin is a hormone produced by the pancreas that helps regulate the flow of blood glucose from the bloodstream into the cells in the body where it can be used as a fuel. Without insulin, blood glucose can build up in the blood and lead to various symptoms and complications, including fatigue, frequent infections, cardiovascular disease, nerve damage, kidney damage, eye damage, and other issues. Individuals with type 1 or type 2 diabetes need to monitor their glucose levels in order to avoid these symptoms and complications.

Analyte monitors, and in particular, glucose monitors for the monitoring of glucose levels for the management of diabetes, are constantly being developed and improved. Although there are several platforms for monitoring analytes such as glucose available on the market, there is still a need to improve their precision, wearability, and accessibility to end-users. In addition, there is a desire to provide robust, less painful, less error-prone, and/or generally more effective continuous glucose monitors which may be attached to the patient's body for a more prolonged period of time, as well as glucose monitor features that can be used together with such improved monitor and monitor designs.

In particular, as continuous glucose monitors improve, for example, as life cycles of monitors have proposed to increase in certain applications and as various different ways have been proposed to provide increased accuracy and decreased discomfort for patients who wear and use such monitors, it has become more difficult to develop sensor members for such continuous glucose monitors that satisfy all of these heightened expectations.

### SUMMARY

Many continuous glucose monitors are intended to be worn on a patient's skin for a duration of multiple days or weeks. Most or all commercially available glucose sensors on the market today sense glucose in interstitial fluid (ISF) below the surface of the skin. Such sensing or monitoring therefore typically involves an initial step of inserting a sensing portion of a sensor member of the glucose monitor under the patient's skin. For the most part, this insertion step will involve puncturing the surface of the skin, for example, with a separate needle, for example, on an applicator to provide access for inserting the sensor. Thereafter, the needle or other sharp may be retracted, while the sensor member stays in place under the patient's skin. The rest of the monitor may be adhered to the patient's skin above the sensor member, and may be physically connected to the sensor member, in order to hold the sensor member in place under the skin, to protect the sensor member from environmental and other external conditions, and to provide and house electronics associated with the analyte monitoring, for example, a battery to provide power to the system, a processor and other circuitry to record and/or analyze electrical signals received from the sensor member, and/or a transmitter to transmit data to an offsite location, for example, a cellular phone or server configured to receive and further process the collected data.

Continuous glucose monitors and sensors need to be very consistently accurate, in order to perform their function of detecting blood glucose levels for a patient seeking to self-medicate with insulin. Such continuous glucose monitors are relied upon to alert the patient of low blood glucose levels, in which case the patient can administer insulin to raise blood glucose levels and alleviate the issue. Therefore, an inaccurate reading could potentially lead to patient harm, for example, failing to alert the patient to administer insulin when the patient's blood glucose levels are low, or improperly alerting the patient of low blood glucose and causing the patient to administer insulin when it is not medically necessary to do so.

In an effort to improve monitoring, reliability, and robustness, continuous glucose sensors have been met with a number of different challenges. Among these challenges have been, for example, restrictions on sensor life and efficacy due to the amount of enzyme available to facilitate the necessary enzymatic reactions associated with monitoring, time delays associated with the measurements taken by the monitors as compared to the actual blood glucose levels of the patient as well as the variability of such delays with respect to the depth and/or other positioning of the sensing regions of the sensor members, natural or other biological responses causing biofouling (e.g., the accumulation of unwanted biological material on the sensor member or surroundings that may cause unwanted deviations in the monitor's efficacy), and/or changes in position of the sensor member due to motion, for example, due to movement by the patient.

In addition, continuous glucose sensors must maintain robust electrical connections between their sensing regions and the rest of the electronics in the housing, with the electrical connections being strong enough to sustain through the life of the sensor, so that data can be properly acquired from the sensing regions. However, these robust electrical connections cannot adversely affect the performance of the sensing regions themselves. So a more effective way to provide both consistent and robust electrical connections has also been sought.

According to embodiments of the invention, sensor members may be fabricated or otherwise manufactured with two or more working electrodes, or two or more sets of electrodes (e.g., with multiple working electrodes and associated reference electrodes and/or counter electrodes). At least the working electrode in multiple electrode arrangements would be equivalent to one another, but would generally function independently from one another. Such multiple electrodes can be arranged in different ways on the sensor member, depending on the desired performance characteristics and features.

For example, in some embodiments, working electrodes may be arranged close to one another, e.g., adjacent, or at a same axial position on the sensor member but on opposite faces, for example. Here, both working electrodes may be monitored, which can for example, allow for a form of error detection when the signals from the electrodes do not match. In some embodiments, the electrodes can be arranged similarly, but with the second electrode covered with additional layers such as a diffusion membrane which allows less glucose through, in order to preserve the enzyme on the second electrode to be utilized later. This arrangement may allow for use of the electrodes in a sequential manner, in order to extend the life of the sensor. In some other embodiments, one of the electrodes may instead not be coated with an active enzyme layer at all, where that particular electrode may instead be used to measure a baseline signal, for example, to correct or otherwise compensate for other biological behavior or other sources of variability on the main electrode that is monitoring the blood glucose levels.

In other embodiments, electrodes may be arranged to be spaced apart along the length of the sensor member, so that when the sensor member is implanted, one electrode or electrode set may be located in the epidermis or the dermis, while another electrode or electrode set may instead be located in the hypodermis or subcutaneous layer. With this arrangement, the electrodes at different depths can be used to compare time delays at the different depths, so that the patient's blood glucose level readings can be more accurately compensated accordingly.

In addition to the above, having a multiple electrode arrangement may also mitigate anomalies associated with compression on the wearable sensor, for example, when the reading from one electrode may be affected by compression of the tissue surrounding the sensing region, but the other electrode is not similarly affected, sensors according to embodiments of the invention may be better equipped to detect such signal artifacts or variances that are induced by pressure or tissue compression.

When multiple electrodes are operated concurrently, in the event a first sensor is identified as no longer functioning, the patient may not need to discard the current system for a new one. Instead, the system could automatically transition to an electrode in the system that is still working and continue taking further measurements without interruption.

According to an embodiment of the invention, a monitor for determining analyte concentrations in vivo includes a housing configured to adhere to a patient's skin, and a sensor member configured to extend from the housing into the patient's skin. The sensor member includes at least a first working electrode and a separate second working electrode that are independently connected to electronics in the housing via separate electrical connections and that function independently from one another.

The first and second working electrodes may both be configured to determine analyte concentrations for the same analyte.

The monitor may further include a single counter electrode and a single reference electrode that are utilized by both the first and second working electrodes.

The monitor may further include a first counter electrode and a first reference electrode utilized by the first working electrode, and a second counter electrode and a second reference electrode utilized by the second working electrode.

The first and second working electrodes may be positioned in close proximity to one another.

The first and second working electrodes may be formed on a same face of the sensor member.

The first and second working electrodes may be spaced apart from one another in a length direction of the sensor member. When the sensor member is implanted in the patient's skin, the first and second working electrodes may be configured to be positioned in different tissue spaces in the patient's skin. The first working electrode may be configured to be positioned in the dermis, and the second working electrode may be configured to be positioned in a layer of the patient's skin that is different from the dermis. The monitor may further include a first counter electrode and a first reference electrode utilized by the first working electrode, and a second counter electrode and a second reference electrode utilized by the second working electrode. The first and second counter electrodes may be connected to one another in series, and the first and second reference electrodes may be connected to one another in series.

The first working electrode may be formed on a first face of the sensor member, and the second working electrode may be formed on an opposite second face of the sensor member.

The first and second working electrodes may be formed on a first face of the sensor member, while a majority of the respective electrical connections for the first and second working electrodes may be formed on an opposite second face of the sensor member.

An enzyme layer may be formed on both the first and second working electrodes.

An enzyme layer may be formed on the first working electrode, but may not be formed on the second working electrode.

A first diffusion layer may be formed on the first working electrode, but may not be formed on the second working electrode.

The first and second working electrodes may be operated concurrently.

The first working electrode may be activated when the monitor is initially applied to the patient, while the second working electrode may be preserved. When the first working electrode fails or otherwise stops working, the monitor may activate the second working electrode to replace the first working electrode.

The monitor may be configured to determine a blood glucose level of the patient.

According to embodiments of the invention, various different arrangements of sensor members with multiple working electrodes or electrode sets can be implemented, and can facilitate improved sensor performance in any of various different ways. For example, sensor members can be arranged to have extended life spans based on sequential usage of the multiple electrode arrangement, for example, if different electrodes are coated with different membranes and/or coatings that allow the electrodes to function for different durations of time. Different depth electrodes can allow for quantification of the time delay relative to one another, for example, in combination with known time delays related to certain tissue spaces, to provide very accurate real time sensing without delays, and here also one electrode or electrode set could also be arranged with a lower diffusion membrane and/or coating that can extend the life of the sensor or more generally improve accuracy of the sensor. An extra electrode or electrode set without enzyme could quantify background noise in real time so that the sensor signal from the working electrode with enzyme could be compensated for the background noise. And even a redundant sensor that is not differently arranged can, for example, allow for higher confidence diagnoses of error state cases from the sensor when the two signals do not align or match. Any or all of these additional benefits can be realized and leveraged to facilitate monitoring and detection of persistent hypoglycemia, or low blood glucose levels.

### BRIEF DESCRIPTION OF THE DRAWINGS

Further features and advantages of the invention will become apparent from the description of embodiments by means of the accompanying drawings. In the drawings:

FIGS. 1A and 1B schematically show a human body with an analyte monitor including an analyte sensor according to embodiments of the invention, where the analyte monitor is attached at different positions on the body.

FIG. 2 shows a perspective view from above an exemplary analyte monitor including an analyte sensor according to embodiments of the invention.

FIG. 3 shows a perspective view from below the analyte monitor of FIG. 2.

FIG. 4 shows an enlarged perspective view of a portion of a sensor member of an analyte monitor according to an embodiment of the invention.

FIG. 5 shows a cutaway view of a portion of a patient's skin with a schematic depiction of a sensor member of an analyte monitor according to an embodiment of the invention implanted therein.

FIG. 6 shows an enlarged view of a portion of a sensor member of an analyte monitor according to a first embodiment of the invention, for example, the sensor member shown in FIG. 4.

FIG. 7 shows an enlarged view of a portion of a sensor member of an analyte monitor according to a second embodiment of the invention.

FIG. 8 shows an enlarged view of a portion of a sensor member of an analyte monitor according to a third embodiment of the invention.

### DETAILED DESCRIPTION

In the following detailed description, only certain embodiments of the subject matter of the present disclosure are described, by way of illustration. As those skilled in the art would recognize, the subject matter of the present disclosure may be embodied in many different forms and should not be construed as being limited to the embodiments set forth herein.

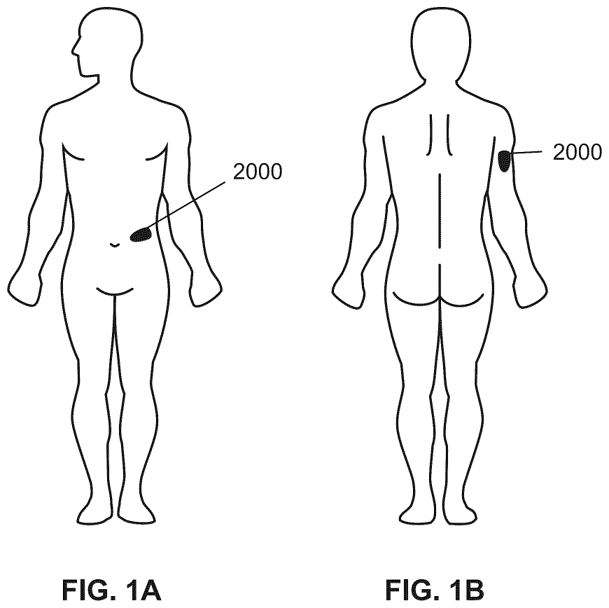

Monitors that include analyte sensors, such as glucose monitors, and in particular continuous glucose monitors, can be attached to a patient's body in different locations, in order to for example, improve glucose monitoring and/or a patient's comfort, since the continuous glucose monitors must remain adhered to the patient's skin, sometimes for a few days or more. FIG. 1A shows a first exemplary analyte monitor 2000 that is adhered to a patient's abdominal region, while FIG. 1B instead shows the exemplary analyte monitor 2000 adhered to a patient's arm. These are only meant to be example adhesion sites, and in other situations, this or a similar analyte monitor may instead be adhered or otherwise attached to other parts of the patient's body.

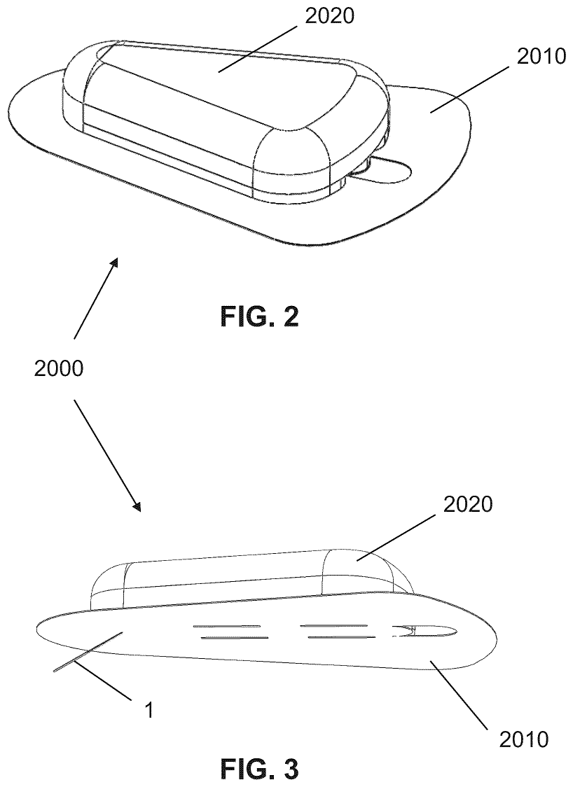

FIGS. 2 and 3 show different schematic views of an example analyte monitor 2000, which can be a continuous glucose monitor, according to an embodiment of the invention. The continuous glucose monitor 2000 may include a base or cradle 2010 that may have an adhesive layer for adhering to a patient's skin, a transmitter 2020 for transmitting data to and/or from a location away from the monitor, and a sensor member 1 which may include an integrated analyte sensing region such as a glucose sensor. It is to be understood that the example analyte monitor shown in FIGS. 2 and 3 are for illustrative and descriptive purposes only, and that analyte monitors with different structures and functionality can also be used in conjunction with the sensor assemblies described below, without departing from the spirit or scope of the invention.

The base 2010 will generally include an adhesive patch or other adhesive mechanism on its lower surface in order to facilitate attachment of the base 2010 to the surface of the patient's skin. In some embodiments of the invention, the sensor member 1 may be integrally formed with the base 2010 so as to be implanted under the patient's skin upon attachment of the base 2010 to the surface of the skin, while in other embodiments the base 2010 may be applied to the skin first, and the sensor member 1 may be advanced to a position where the sensor member 1 becomes attached to the base during or after implantation of part of the sensor member 1 under a patient's skin. For some embodiments, application of the base 2010 and/or the sensor member 1 may be facilitated with one or more applicators.

In some embodiments, after the base 2010 has been attached to a surface of the patient's skin and after at least part of the sensor member 1 has been advanced under the patient's skin, the transmitter 2020 which may be a separate part can be attached to the base. In some embodiments, the transmitter 2020 may include, for example, a power source such as a battery, while the base 2010 and/or the sensor member 1 may include additional electrical circuitry or contacts to complete a circuit, such that the monitor 2000 is powered up upon assembly of the transmitter 2020 to the base 2010. Other embodiments may include other arrangements, for example, where the battery is housed in the base 2010 instead of on the transmitter 2020, etc. Still other embodiments may include further different arrangements, for example, an integrally manufactured monitor where the base and transmitter are formed together in a single main body and which may not be separable from one another by the end user, where such embodiments may also include an integrated sensor member, or may include a sensor member that is implanted through the main body of the monitor after the main body has been adhered to the surface of the patient's skin.

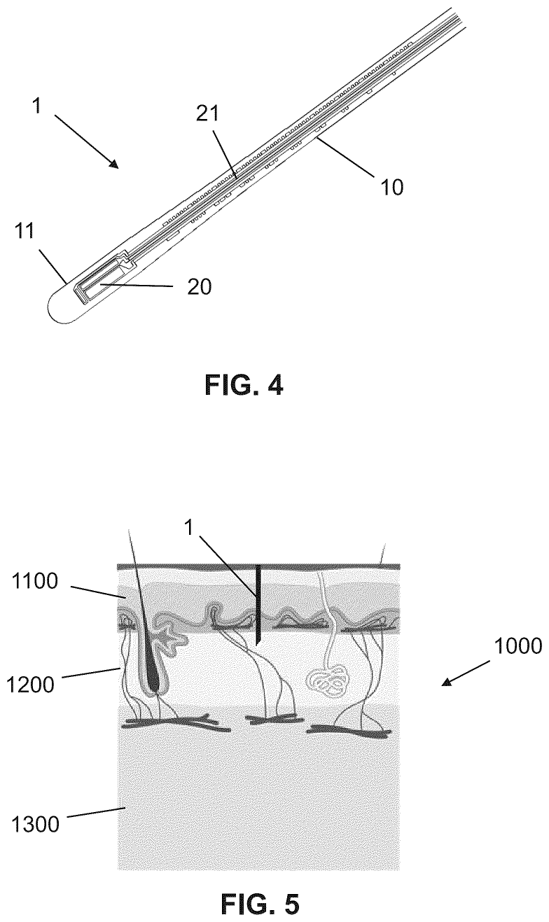

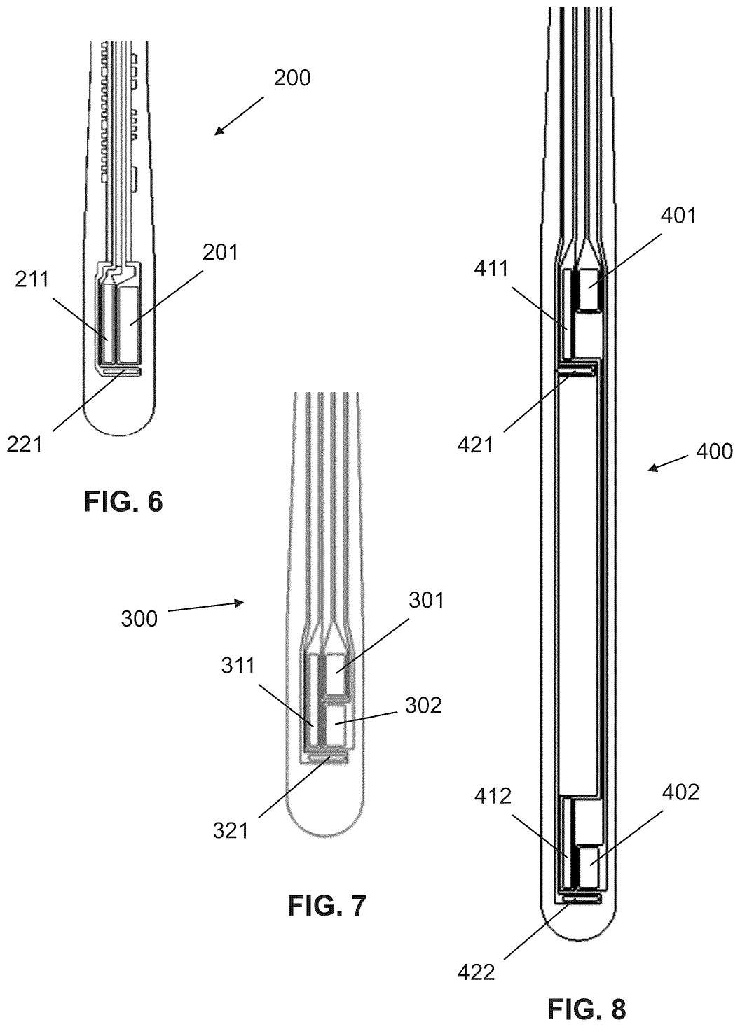

Construction of an example sensor member 1 according to an embodiment of the invention will now be discussed in greater detail, with reference to FIG. 4. As seen in FIG. 4, a sensor member 1 may include an implantable portion that includes a main body 10 and a sensing region including one or more sensing electrodes 20. The sensing electrodes 20 may include one or more of a working electrode, a counter electrode, or a reference electrode, where other embodiments may include more or less electrodes, according to the particular application. Specifically, according to some embodiments of the invention, at least more than one working electrode may be incorporated into the sensor member. In other embodiments, multiple working electrodes may be implemented with their own respective reference electrodes and/or counter electrodes. The working electrodes or electrode sets may be on a same face of the sensor member 1, or may be on opposite faces of the sensor member. In some embodiments, the working electrodes or electrode sets may further be spaced apart along an axis of extension of the sensor member. More arrangement variations are described in greater detail below, with reference to FIGS. 6-8.

The main body 10 may include a sensor tip or distal end region 11, where the sensing electrodes are typically located at or near the distal end region 11. In some embodiments, the distal end region 11 may be enlarged, for example, in a width direction compared to other portions of the main body. For example, in one embodiment, the distal end region 11 may be substantially teardrop-shaped or otherwise widened with a tapering profile. In other embodiments, the distal end region may include arms or teeth extending laterally outwardly, or may include other shapes or profiles that provide for a varying width. Enlarging the distal end region 11 can help anchor the sensing electrodes 20 at a particular desired position under the surface of the skin and facilitate improved positioning and retention of the sensor member 1 under the skin after implantation. The sensing electrodes 20 may further be electrically connected to leads 21 that extend along the main body 10 of the sensor and to an opposite proximal end of the sensor member 1, to facilitate electrical connectivity and communication of the sensing electrodes 20 with other electrical components of the monitor.

FIG. 5 shows a cutaway view of a portion of a patient's skin, with a schematic depiction of the sensor member 1 implanted therein, according to an embodiment of the invention. As shown in FIG. 5, the skin 1000 of a human includes various layers, such as an epidermis 1100 that is closest to the surface of the skin, an intermediate dermis 1200, and a deeper hypodermis or subcutaneous layer 1300. Sensor members 1 according to embodiments of the invention may only extend into the shallow regions of the dermis 1200, with sensing regions in either the shallow regions of the dermis 1200, the epidermis 1100, or both, rather than extending more deeply into the hypodermis 1300, as is the case with many other monitors on the market. Positioning the sensing regions of the sensor member 1 in the shallow regions of the dermis 1200 or the epidermis 1100 may have many advantages. For example, the smaller size of the sensor member 1 according to embodiments of the invention, both in terms of length and thickness, may reduce trauma during insertion and/or discomfort during the life of the sensor/monitor. The reduction in size of the sensor member 1 compared to traditional sensor members, or more generally, the reduced depth to which the sensor member 1 extends under the skin, will also allow sensing in less fatty layers of the skin, consequently resulting in faster glucose transport, more accurate signal readings by the sensor member, and a shorter delay or time lag associated with being able to retrieve useable signals from the sensor member 1. Targeting the shallower regions of the dermis 1200 furthermore allows for some tolerance or leeway, where for example, if a sensor member 1 is implanted at a slightly more vertical angle than intended, the sensing regions of the sensor member 1 should still be positioned in the dermis 1200 rather than deeper in the hypodermis 1300, and still benefit from both reduced discomfort and less glucose transport delays due to fat in the tissue.

Sensor members in other embodiments may be positioned shallower or deeper than as described, without departing from the spirit or scope of the invention. For example, as has been previously discussed, some embodiments of the invention may incorporate multiple working electrodes or electrode sets or bundles that are spaced apart along a length or axis of extension of the sensor member 1. In such embodiments, the sensor member 1 may be arranged to be longer, for example, configured to extend into the hypodermis 1300. However, at least one working electrode or electrode set may be arranged more proximally or shallower on the sensor member 1, so as to be positioned in the epidermis 1100 or shallow dermis 1200, for example, when the sensor member 1 is fully implanted, while a further electrode or electrode set may be arranged more distally or deeper on the sensor member 1, so as to be positioned in the hypodermis 1300, for example, when the sensor member 1 is fully implanted. Such an arrangement may allow for collection of different types of signals, which may be beneficial in some signal processing cases or situations, as described in greater detail below.

Referring now to FIG. 6, FIG. 6 shows an enlarged view of a portion of a sensor member of an analyte monitor according to a first embodiment of the invention. The sensor member 200 shown in FIG. 6 may be similar to the sensor member 1 in FIG. 4 in some respects. The portion shown may be a distal end portion of the sensor member 200. The sensor member 200 in FIG. 6 may include a first working electrode 201, a first counter electrode 211, and a first reference electrode 221. The working electrode 201 may work together with the counter electrode 211 and the reference electrode 221 to generate signals that can be processed to determine blood glucose levels of the patient in which the sensor member 200 is implanted. The working electrode 201, the counter electrode 211, and/or the reference electrode 221 according to embodiments of the invention may be fabricated or otherwise manufactured, for example, by leveraging improved photolithography and/or other compatible nano-manufacturing technology methods, and may be arranged for example, with a layered filament design. While not visible in FIG. 6, in some embodiments, a second set of electrodes, for example, at least a second working electrode, which may further be grouped with a second counter electrode and/or a second reference electrode, may be formed on an opposite face of the sensor member 200 (e.g., on a side of the sensor member 200 that faces the paper in FIG. 6). Such a second set of electrodes may be manufactured or otherwise formed to have an arrangement similar to the arrangement of the visible electrode set shown in FIG. 6, or may be arranged differently in some embodiments. The two sets of electrodes may further be electrically isolated from one another (e.g., they may collect data completely separately from one another), or they may be in direct electrical communication with one another. During manufacturing, the two working electrodes or electrode sets can be manufactured independently, where the separate electrodes or electrode sets can be fabricated separately and positioned on opposite sides of the filament or substrate, for example, via two separate sets of fabrication steps, to facilitate placing electronics on both sides of the filament. Some embodiments may utilize vias or other electrical means to facilitate communication between the electronics on either side of the sensor member 200.

In other embodiments, the electrodes or electrode sets may be on a first side of the sensor member, while as much of the electrical connections may instead be fabricated on the opposite side (not shown in the figures). Generally, electrical connections and leads cannot have layers on top or be covered by layers or coatings that are required for the electrodes at the sensing regions to measure glucose, since for example, the additional layers may affect the electrical conductivity of the electrical connections. Because of this restriction, fabrication or other manufacturing is typically complicated by the need for additional masks and/or other methods and steps to form keep out zones, in order to cover the sensing regions with the necessary enzyme and/or membrane layers while omitting those same layers from the electrical connections. By arranging the electrodes on the opposite side or face of the sensor member from as much of the electrical connections as possible during fabrication, for example, when manufactured via improved photolithography and/or other compatible nano-manufacturing technology methods, the manufacturing process can then focus on delivering consistent layers of enzyme and/or membrane layers on the sensing regions, without limitations around their placement previously caused by adjacent electrical connections. Instead, the entire planar surface that houses the electrodes, and the sensing region in general, can receive the same fabrication treatments, which may remove barriers that cause inconsistency, such as keep out zones.

Such improvement and simplification in manufacturing would allow highly consistent processing within a given batch of wafers on which the sensor members are fabricated, as well as between different batches of wafers. Such improved consistency can be leveraged, for example, into more cost effect, consistent, higher yield, and more accurate sensor members overall. In some cases, high enough accuracy and consistency may even lead to the removal of the need for sensor calibration. For example, existing sensor members are manufactured such that each sensor member or each batch of sensor members must be independently measured or tested to quantify performance and to provide a factory calibration value to the user, or the user must perform additional steps such as providing their blood glucose estimate to the system, in order to properly calibrate the sensor member before receiving data. In contrast, in cases where consistently in manufacturing sensor members is sufficiently improved, user calibration steps may be simplified and/or eliminated altogether, which would simplify end user steps and remove or at least reduce reliance on the end user for proper sensor functionality. Such “no calibration” sensor members would allow for the reduction in infrastructure, as well as reduce the risk of user error. Such technological improvements can also generally reduce the cognitive burden on patients associated with managing their diabetes monitoring, which generally brings added value to patients.

FIG. 7 shows an enlarged view of a portion of a sensor member of an analyte monitor according to a second embodiment of the invention. The portion of sensor member 300 shown in FIG. 7 may be a distal end portion of the sensor member 300.

As seen in FIG. 7, the set of electrodes on sensor member 300 is arranged similarly to the set of electrodes on sensor member 200 in FIG. 6. The sensor member 300 includes a similarly situated counter electrode 311 and a similarly situated reference electrode 321. However, instead of one working electrode, the sensor member 300 incorporates two separate working electrodes 301, 302. In the arrangement shown, the working electrodes 301, 302 occupy a similarly sized footprint as the working electrode 201 on the sensor member 200 in FIG. 6, and are therefore constructed to be about half the size of the working electrode 201. The working electrodes 301, 302, may be functionally equivalent but function independently from one another, and may include separate respective leads to facilitate separate communication between the working electrodes 301, 302, and the rest of the electronics package of the analyte sensor, for example, housed in the main body or housing adhered to the surface of the patient's skin. Separate construction of the working electrodes 301, 302 also ensures that the working electrodes 301, 302 can properly function independently from one another. In other arrangements, the working electrodes 301, 302 may be arranged in series or in parallel, for example, depending on the particular needs and/or objectives of the sensor member.

As illustrated, the sensor member 300 includes one counter electrode 311 and one reference electrode 321 that work together with the two working electrodes 301, 302, to monitor analyte levels such as blood glucose levels in the patient. In other embodiments, there may also be more than one counter electrode and/or reference electrode. For example, in some embodiments, there may be a separate corresponding counter electrode for each working electrode, which for the example shown in FIG. 7, may be arranged such that each working electrode and corresponding counter electrode are positioned adjacent to one another in a width direction. Such additional electrode arrangements may also be wired independently, in series, or in parallel, or may also be otherwise arranged. Still other arrangements or electrodes and/or electrode sets may also be incorporated without departing from the spirit or scope of the invention.

As shown in FIG. 7, the sensor member 300 includes working electrodes 301, 302 that are adjacent to one another, e.g., close enough that they would likely be positioned in the same tissue space when the sensor member 300 is implanted in the patient. For example, in cases where the sensor member is configured to extend into the dermis of the patient, both working electrodes 301, 302 may be positioned together in the dermis of the patient's skin. In other embodiments, the sensor member may be sized and arranged to hold the electrodes or electrode sets in different layers or depths in the patient's skin. In still other embodiments (see, e.g., sensor member 400 in FIG. 8, described in greater detail below), working electrodes may instead intentionally be spaced apart and configured to be positioned in multiple different tissue spaces.

In one arrangement, particularly when the working electrodes 301, 302 are arranged similarly and coated similarly, both of the working electrodes may be concurrently operated, which can allow for a form of error detection, for example, when an anomaly or inconsistency arises in one of the signals and/or when the signals from the electrodes generally do not otherwise match or fall within a certain deviation from one another. Another potential benefit may be redundancy, for example, where the sensor system may not need to be discarded or replaced in the event that one working electrode fails or otherwise stops working. If a second working electrode is present in the system, the system can then override or ignore the signals from the first working electrode if the first working electrode is determined to be working improperly, and only monitor the patient's blood glucose levels using the second working electrode that is functioning properly without interruption. In another arrangement, the working electrodes 301, 302 may be prepared slightly differently, for example, with one of the working electrodes (e.g., working electrode 302) coated or otherwise supplied with an enzyme such as glucose oxidase, while the other working electrode (e.g., working electrode 301) is not coated or supplied with glucose oxidase. In such an arrangement, a differential may be observed, where readings from a first working electrode includes readings attributable to both the patient's blood glucose levels as well as other biological reactions and responses that occur near the sensor member, while readings from a second working electrode includes baseline readings that may be attributable to only the non-glucose related biological reactions, such that the system can collect and compensate or adjust the readings from the first working electrode with the readings from the second working electrode, potentially improving the accuracy of the patient's blood glucose measurements.

FIG. 8 shows an enlarged view of a portion of a sensor member of an analyte monitor according to a third embodiment of the invention. The portion of sensor member 400 shown in FIG. 8 may be a distal end portion of the sensor member 400.

Compared to the previous sensor members 200, 300 described above, the sensor member 400 is more elongate in the length direction. In addition, the sensor member 400 includes two separate sets of electrodes, with a more proximal or shallower electrode group or set including a first working electrode 401, a first counter electrode 411, and a first reference electrode 421, and a more distal or deeper electrode group or set including a second working electrode 402, a second counter electrode 412, and a second reference electrode 422. An axial gap is formed between the proximal and distal electrode sets, the gap being configured based on the desired positioning of the electrode sets when the sensor member 400 is inserted into the patient's skin. For example, when the shallower electrode set is desired to be positioned in the dermis, while the deeper electrode set is desired to be positioned in the hypodermis or subcutaneous layer, the size of the gap is selected to be a sufficient distance to facilitate the proper positioning of the respective electrode sets in their respective associated skin layers.

In addition, in sensor member 400, while the working electrodes 401, 402 have separate electrical leads or traces, and can therefore function independently from one another, the counter electrodes 411, 412 are connected in series, and the reference electrodes 421, 422 are connected in series as well. In other embodiments, other electrode connection arrangements can also be implemented without departing from the spirit or scope of the invention. For example, in another embodiment, the counter electrodes 411, 412 may also have their own respective separate electrical connections and function independently from one another.

When employing a sensor member similar to sensor member 400 in FIG. 8, with working electrodes or electrodes sets being positioned in different tissue spaces or different depths within a same tissue space, the signals from the respective working electrodes can be used to compare tau or time delays at the different depths compared to a patient's real-time blood glucose levels, so that the patient's blood glucose level readings can be more accurately compensated accordingly. Generally, subcutaneous blood glucose measurements have a delay on the order of about 15 minutes or less, while dermal blood glucose measurements have a much shorter delay on the order of about 2 minutes. Monitoring both readings from the patient can help account or compensate for these time delays, and provide better predictors or estimates in real time. Since the general differential in response times and delays for different tissue spaces are known, when readings are obtained from different tissue spaces, the different data from the two working electrodes can be compared and utilized to better compensate for time delays, and can therefore be adjusted to reduce the effects of such time delays on data being delivered to the patient during active monitoring. Based on these improvements, systems according to embodiments of the invention can more easily and/or more readily facilitate identification and measurement of changes in tau, or the time delay between real in-vivo blood glucose and the measurement taken by the sensor.

Importantly, at least one of the working electrodes should be positioned in the dermis, or at the very least, shallower than the traditional subcutaneous placement of most sensing regions on the market. According to embodiments of the invention, with one working electrode positioned in the dermis, time delays for blood glucose readings compared to real time blood glucose levels were reduced to less than 2 minutes for about 93% of the readings, compared to top competitor devices who only achieved time delays of less than two minutes about 39% of the time. Similarly, the tau or time delays for blood glucose readings was measured to be less than 5 minutes 99% of the time according to embodiments of the invention, compared to only 69% of the time for top competitor devices. Therefore, when utilizing sensors according to embodiments of the invention, with at least one sensing region positioned in the dermis, the readings from the dermal sensing region can be leveraged to provide blood glucose data to patients that are delayed by much less time, compared to competitor devices. If another sensing region is positioned in another layer of the patient's skin, for example, in the subcutaneous layer, the two different types of data can be concurrently measured, tracked, compared, and processed in various different ways to provide even further useful information to patients.

In addition, the time constant or time constants can be more accurately measured and determined based on the differential between the signals from the working electrode in the dermal layer and the signals from the working electrode in the subcutaneous layer to make a more accurate measurement of the patient's blood glucose level. As such, the system according to embodiments of the invention may be capable of self-calibrating by utilizing the signals from the two different working electrodes, without any user input or contribution. For example, initially a population based time constant estimate may be used (e.g., for each working electrode) upon initial starting up of the analyte sensor, e.g., before the initial calibration process has been completed. The system can then start gathering data retrieved from the two tissue layers, for example, after a first glycemic excursion, and then leverage the fluctuations and delays in the signals to refine the calibration and adjust the time constants based on the differentials observed between the signals collected from the two different tissue layers. Thereafter, the accuracy of the time constants may continue to be refined and fine-tuned over time, for example, as more glycemic excursions and relative delay and time constant data and history becomes available.

Another potential benefit to monitoring in different tissue spaces may be the ability to better mitigate anomalies associated with compression on the wearable sensor or on the tissue surrounding the sensor member. In some cases, sensitivity to a compression event are different in different tissue spaces, so the signals collected from the different tissue spaces, and their associated changes upon a compression event, may be utilized to better detect and adjust the data in such cases. As such, this type of sensor arrangement may be better equipped to detect signal artifacts or variances that may have been induced by pressure or tissue compression.

Yet another potential benefit of a dual depth arrangement is a better ability to detect persistent hypoglycemia without sacrificing performance. For example, the sensing regions of sensors may preferably be positioned in the dermis of the patient, where shorter time delays can be leveraged to provide blood glucose data that is closer to real-time data when compared to sensing regions positioned in the hypodermis or subcutaneous region. However, sensing regions positioned in the subcutaneous region have a better ability to detect instances of persistent hypoglycemia. Therefore, having sensing regions in both tissue spaces can be beneficial, since the system can, for example, utilize both the more current blood glucose data from the sensing region in the dermis, while also being able to more readily detect instances of persistent hypoglycemia by also monitoring the data from the sensing region in the subcutaneous layer.

While not pictured in detail, sensor members according to some embodiments of the invention, including the sensor members 200, 300, 400 in FIGS. 6 to 8, can be formed on a thin flexible substrate, to facilitate less trauma to the surrounding tissue and less discomfort to the patient both during insertion of the sensor member and while the sensor member is held under the patient's skin. The sensor member may further be fabricated or otherwise manufactured in layers, for example, with a flexible base or substrate, electrodes and electrical/conductive connections and other materials to facilitate electrical communication with the wearable electronics package housed in the main body of the analyte sensor, enzymatic material to facilitate the electrochemical reactions necessary for a desired type of analyte monitoring, a diffusion limiting membrane, and/or a hydrophilic outside layer. More or less other layers may also be incorporated, based on the particular design of the sensor member.

In the case of blood glucose monitoring, the enzymatic layer or material coated onto the sensing regions of the sensor member may be glucose oxidase. In some embodiments, the glucose oxidase may be coated or otherwise supplied to all of the electrodes similarly, while in other embodiments, less than all of the electrodes, for example, only one of the working electrodes, may be coated or otherwise supplied with glucose oxidase, while at least one other working electrode may purposefully be prepared and implanted without any active enzyme. Such a non-enzymatic working electrode may be used, for example, to generate signals caused by other biological behavior in the tissue surrounding the sensor member that is not related to glucose, so that for example, the system can compensate the glucose measurements from the main working electrode with these secondary or auxiliary data readings.

Some other sensors on the market have tried to employ a working electrode connected to multiple locations in series on their respective sensor members, in an effort to, for example, obtain an average signal from multiple tissue spaces. However, electrode arrangements according to embodiments of the invention provide a new way to obtain multiple sets of data that can then be applied to active logic decisions, in order to provide the best possible data to the patient.

In addition, in cases where a diffusion limiting membrane is used, the diffusion limiting membrane may in some embodiments only selectively be applied to one or some of the working electrodes, while at least one additional working electrode may not be coated with the diffusion limiting membrane, a thinner layer of the diffusion limiting membrane, or with a diffusion limiting membrane that facilitates higher diffusion. With such an arrangement, a first working electrode exposed to higher diffusion may operate as the main working electrode at the start of the life cycle of the analyte sensor. When the first working electrode starts to wear down, for example, due to depletion of enzyme from reactions with glucose over time and/or due to increased exposure to biofouling, among other factors, a second working electrode that is exposed to a lower diffusion and therefore better preserved, e.g., by allowing less glucose through the diffusion layer to preserve enzyme over time as well as reduce biofouling, can lead to the second working electrode exhibiting a prolonged life span compared to the first working electrode, and thus may allow the analyte sensor to continue operating for a longer duration if system switches to utilizing the second working electrode as the main data collection electrode. Such an arrangement may provide for a novel way to achieve a longer life for the analyte sensor as a whole. In such arrangements, the system can still also compensate for the different diffusion layer characteristics during signal processing, so that signals from all of the working electrodes employed can still be collected concurrently and utilized together to calculate accurate blood glucose levels of the patient. In yet another potential arrangement, the second working electrode or electrode set may be preserved completely, for example, the second working electrode may not be electrically activated until a later time, for example, upon detecting that the first working electrode has failed or otherwise stopped working, which may extend the overall life of the analyte sensor even more. This would prolong the life of most analyte sensors, since the biggest limiting factor to most analyte sensors is sensor life and sensor failure.

Traditionally, measures that have been taken to try to extend the life spans of sensor members of analyte sensors included, for example, polyvinyl alcohol (PVA) coatings and slower diffusion membranes. However, such arrangements cause delays in measurements relative to the actual blood glucose levels of the patient, which may for example, delay treatments in the case of a low blood glucose indication by the system. Other systems have also tried to use a “look forward” algorithm, but such algorithms are based on population data and applied universally, instead of a calibrated comparison more accurately based on data of the specific patient, and therefore is also less accurate. According to embodiments of the invention, a first working electrode can function with less restrictions (e.g., without or with a less prohibitive diffusion layer), and can therefore provide more accurate blood glucose readings with less time delays, while a second working electrode can assume the primary monitoring duties when the first working electrode is nearing end of life.

In addition to the embodiments that have already been described above, it is also possible to combine embodiments, e.g., different features from the various described embodiments, to provide even more different variations of analyte monitors, without departing from the spirit or scope of the invention. In addition, the inventions should not be limited to the structures and/or shapes described in the embodiments above.

While the subject matter of the present disclosure has been described in connection with certain embodiments, it is to be understood that the subject matter of the present disclosure is not limited to the disclosed embodiments, but, on the contrary, the present disclosure is intended to cover various modifications and equivalent arrangements included within the spirit and scope of the appended claims, and equivalents thereof.

What is claimed is:

1. A monitor for determining analyte concentrations in vivo, comprising:
a housing configured to adhere to a patient's skin; and
a sensor member configured to extend from the housing into the patient's skin, wherein the sensor member comprises at least a first working electrode and a separate second working electrode that are independently connected to electronics in the housing via separate electrical connections and that function independently from one another.

2. The monitor of claim 1, wherein the first and second working electrodes are both configured to determine analyte concentrations for the same analyte.

3. The monitor of claim 1, further comprising a single counter electrode and a single reference electrode that are utilized by both the first and second working electrodes.

4. The monitor of claim 1, further comprising a first counter electrode and a first reference electrode utilized by the first working electrode, and a second counter electrode and a second reference electrode utilized by the second working electrode.

5. The monitor of claim 1, wherein the first and second working electrodes are positioned in close proximity to one another.

6. The monitor of claim 1, wherein the first and second working electrodes are formed on a same face of the sensor member.

7. The monitor of claim 1, wherein the first and second working electrodes are spaced apart from one another in a length direction of the sensor member.

8. The monitor of claim 7, wherein when the sensor member is implanted in the patient's skin, the first and second working electrodes are configured to be positioned in different tissue spaces in the patient's skin.

9. The monitor of claim 8, wherein the first working electrode is configured to be positioned in the dermis, and the second working electrode is configured to be positioned in a layer of the patient's skin that is different from the dermis.

10. The monitor of claim 7, further comprising a first counter electrode and a first reference electrode utilized by the first working electrode, and a second counter electrode and a second reference electrode utilized by the second working electrode.

11. The monitor of claim 10, wherein the first and second counter electrodes are connected to one another in series, and the first and second reference electrodes are connected to one another in series.

12. The monitor of claim 1, wherein the first working electrode is formed on a first face of the sensor member, and the second working electrode is formed on an opposite second face of the sensor member.

13. The monitor of claim 1, wherein the first and second working electrodes are formed on a first face of the sensor member, while a majority of the respective electrical connections for the first and second working electrodes are formed on an opposite second face of the sensor member.

14. The monitor of claim 1, wherein an enzyme layer is formed on both the first and second working electrodes.

15. The monitor of claim 1, wherein an enzyme layer is formed on the first working electrode, but is not formed on the second working electrode.

16. The monitor of claim 1, wherein a first diffusion layer is formed on the first working electrode, but is not formed on the second working electrode.

17. The monitor of claim 1, wherein the first and second working electrodes are operated concurrently.

18. The monitor of claim 1, wherein the first working electrode is activated when the monitor is initially applied to the patient, while the second working electrode is preserved.

19. The monitor of claim 18, wherein when the first working electrode fails or otherwise stops working, the monitor activates the second working electrode to replace the first working electrode.

20. The monitor of claim 1, wherein the monitor is configured to determine a blood glucose level of the patient.
